(19) United States
(12) Patent Application Publication
(10) Pub. No.: US 2020/0093856 A1
(43) Pub. Date: Mar. 26, 2020

(54) Hydrogen-containing composition for ameliorating depressive symptom

(71) Applicant: MiZ Company Limited, Kanagawa (JP)

(72) Inventors: Masatsugu Saito, Kanagawa (JP); Fumitake Satoh, Kanagawa (JP); Yusuke Ichikawa, Kanagawa (JP); Shinichi Hirano, Kanagawa (JP)

(21) Appl. No.: 16/556,789

(22) Filed: Aug. 30, 2019

(30) Foreign Application Priority Data
Sep. 3, 2018 (JP) 2018-164456

(51) Int. Cl. A61K 33/00; A61P 25/24; A61K 9/00

(52) U.S. Cl. CPC A61K 33/00; A61K 9/007; A61P 25/24

(57) ABSTRACT

The present application provides a composition for improving depressive symptoms in a human, comprising hydrogen as an active ingredient, and a method for improving depressive symptoms in a human having depressive symptoms, comprising administering the composition to the human.

### TECHNICAL FIELD

The present invention relates to a composition for improving (or ameliorating) depressive symptoms, comprising hydrogen (hereinafter, also referred to as hydrogen gas, molecular hydrogen, gaseous hydrogen, or hydrogen molecule) as an active ingredient.

The present invention also relates to a method for improving (or ameliorating) depressive symptoms in a human having depressive symptoms, comprising administering the composition to a human.

### BACKGROUND ART

The term “depressive symptom(s)” refers to a depressive state in psychiatric medicine, and includes depression. Causes of depressive symptoms include exogenous factors (somatogenic factors), endogenous factors, and psychosomatic factors (dispositional and environmental factors). In the American Society of Neurological Medicine, depression is categorized as mood disorder, and classified into major depressive disorder (MDD) and dysthymic disorder according to the degree of severity. In Japan, the number of patients with mood disorder tends to increase, which has significant social and economic impacts. Drug therapy is generally used for treatment of depressive symptoms, but has the problem in that a drug is apt to produce adverse reactions. Depression is described in, for example, Non-patent Literature 1.

One of the causes of depressive symptoms is thought to be a reduction in activity of neurotransmitters in the brain, such as serotonin and noradrenalin, but different causes are conceivable. The causes of depressive symptoms have not been elucidated sufficiently.

In such circumstances, the amounts of inflammatory stress biomarkers and oxidative stress biomarkers have been found to increase in MDD, and therefore it has been pointed out that oxidative stress and inflammatory stress are related to neuroprogression of the central nervous system in MDD (Non-Patent Literature 2).

Through experiments mainly with disease model animals such as mice and rats, it has been pointed out that hydrogen has preventing action on disorders from in vivo oxidative stress caused by radical oxygen species (ROS), and hydrogen therapy may be effective against diseases caused partially by oxidative stress. For example, an improvement effect of hydrogen water has been estimated in clinical trials in which hydrogen water is administered to human patients with Parkinson's disease, one of the neurodegenerative diseases (Non-Patent Literature 3 and Non-Patent Literature 4).

In connection with depressive symptoms, a study on the effect of hydrogen water on the depressive behaviors of mouse models has been reported. The report indicates that hydrogen water suppresses activation of inflammasome which is a complex of proteins involved in inflammation and apoptosis (Non-Patent Literature 5).

### PRIOR ART LITERATURE

### Non-Patent Literature

[Non-Patent Literature 1] Norito Kawakami, Journal of Clinical and Experimental Medicine (IGAKU NO AYUMI), 219(13): 925-929 (2006) (Jp)

[Non-Patent Literature 2] N. Bakunina et al., Immunology, 144: 365-373 (2015)

[Non-Patent Literature 3] Shigeo Ohta, Japanese Biochemical Society Proceedings, 87(1): 82-90 (2015) (Jp)

[Non-Patent Literature 4] A. Yoritaka et al., Mov. Disord., 28: 836-839 (2013)

[Non-Patent Literature 5] Y. Zhang et al., Scientific Reports, 6: 23742; DOI: 10.1038/srep23742 (2016)

### SUMMARY OF THE INVENTION

### Problem to be Solved by the Invention

An object of the present invention is to provide a substance enabling improvement of depressive symptoms and having higher safety (e.g. weak adverse reactions).

Drugs for mental diseases such as antidepressant drugs generally have (strong) adverse reactions, and thus improvement of the QOL (quality of life) of patients is one of challenges.

### Means for Solution of Problem

Here, the present inventors have found that by administering hydrogen to a human (e.g. a patient) having depressive symptoms (e.g. through pulmonary administration), the depressive symptoms can be improved (or ameliorated).

Accordingly, the present invention includes the following characteristics.

[1] A composition for improving depressive symptoms in a human, comprising hydrogen as an active ingredient.

[2] The composition according to [1] above, wherein the depressive symptoms are caused by a disease selected from the group consisting of depression, bipolar disorder, dysthymic disorder, anxiety disorder, social anxiety disorder, panic disorder, obsessive-compulsive disorder, autism spectrum disorder, ADHD, sleep disorder, cerebral infarction, dementia, alcohol dependence, and an adverse reaction to a therapeutic agent.

[3] The composition according to [1] or [2] above, wherein the composition is in the form of a hydrogen gas-containing gas.

[4] The composition according to [3] above, wherein the hydrogen gas-containing gas has a hydrogen concentration of 5 to 10% by volume.

[5] The composition according to any one of [1] to [4] above, wherein the composition is administered to a human by pulmonary administration.

[6] The composition according to [5] above, wherein the pulmonary administration is performed in an atmospheric-pressure environment or in a high-pressure environment at 1.02 to 7.0 atm.

[7] The composition according to any one of [1] to [6] above, wherein the composition is prepared in situ using a hydrogen gas generating apparatus at the time of administering the composition to the human.

[8] The composition according to [1] to [7] above, wherein the composition is administered in combination with a therapeutic drug(s) for the depressive symptoms.

[9] The composition according to [8] above, wherein the therapeutic drug(s) is an antidepressant drug or a mood-stabilizing drug.

[10] A method the method comprising administering to the human the composition according to any one of [1] to [9] above.

### Effect of the Invention

According to the present invention, it is possible to improve depressive symptoms by administering hydrogen to a human. Hydrogen is said to have almost no adverse reactions, and is therefore expected to reduce the dosage and the dosing frequency of a drug for mental disease such as an antidepressant drug in a patient having depressive symptoms.

### MODES FOR CARRYING OUT THE INVENTION

The present invention will be described in further detail.

1. Depressive Symptoms and Diseases with Depressive Symptoms

As used herein, the term “depressive symptoms” refers generally to mental disorder characterized by two or more symptoms selected from depressed mood, lowering of motivation, interest and mental activity, impatience, loss of appetite, sleeplessness, persistent sadness and anxiety, and the like, and the degree (degree of severity) of the disorder may be any of “mild”, “moderate” and “severe”. The human having depressive symptoms is not limited to a patient in need of treatment, and may be a human not requiring treatment (e.g. a human having mild disorder which has not been treated yet).

Since it is suggested that oxidative stress and inflammatory stress may be involved in neuroprogression in depression (MDD) as described in N. Bakunina et al., Immunology, 144: 365-373 (2015), hydrogen therapy with hydrogen gas according to the present invention is considered to play some role in improvement of depressive symptoms by reduction of the stresses.

In the present invention, the “depressive symptoms” are not limited to endogenous depressive symptoms, and include exogenous depressive symptoms, and dispositional and environmental depressive symptoms.

The endogenous depressive symptoms are typical depression, and include “mood disorder” in the mental disorder (disease) classification by the American Society of Neurological Medicine, i.e. “dysthymic disorder” which is low in degree of severity and persistent and “major depressive disorder (MDD)” which is high in degree of severity. The endogenous depressive symptoms include bipolar disorder (also referred to as “manic-depressive disease”).

Examples of the exogenous depressive symptoms include depressive symptoms caused by disease or disorder such as dementia, cerebral infarction, hypothyroidism, developmental disorder (e.g. autism spectrum disorder, ADHD (attention deficit/hyperactivity disorder), etc.), alcohol dependence or a therapeutic drug (e.g. interferon) adverse reaction.

The psychosomatic depressive symptoms are disposition- or environment-related depressive symptoms, and examples thereof include anxiety disorder, adaptation disorder, social anxiety disorder, panic disorder, obsessive-compulsive disorder and sleep disorder.

### 2. Hydrogen-Containing Composition for Improving Depressive Symptoms

A first aspect of the present invention provides a composition for improving depressive symptoms in a human, the composition comprising hydrogen as an active ingredient.

The depressive symptoms include symptoms caused by the disease, disorder or symptoms exemplified above, such as depression excluding schizophrenia, dementia, bipolar disorder, anxiety disorder, dysthymic disorder, panic disorder, obsessive-compulsive disorder, autism spectrum disorder, ADHD, sleep disorder, cerebral infarction, alcoholic dependence or an adverse reaction to a therapeutic drug.

Herein, the “hydrogen” which is an active ingredient of the composition of the present invention is molecular hydrogen (i.e. gaseous hydrogen), and is referred to simply as “hydrogen” or “hydrogen gas” unless otherwise specified. As used herein, the term “hydrogen” refers to hydrogen represented by the molecular formula: H₂, D₂ (deuterium) or HD (hydrogen deuteride), or a mixed gas thereof. D₂ is expensive, and is known to have superoxide scavenging effect higher than that of H₂. Hydrogen usable in the present invention is H₂, D₂ (deuterium) or HD (hydrogen deuteride), or a mixed gas thereof, preferably H₂. Alternatively, D₂ and/or HD may be used instead of H₂ or in combination with H₂.

The composition of the present invention is preferably in the form of a hydrogen gas-containing gas.

The hydrogen gas-containing gas is preferably air containing hydrogen gas, or a mixed gas containing hydrogen gas and oxygen gas. The concentration of hydrogen gas in the hydrogen gas-containing gas is more than zero (0) and not more than 18.5% by volume, e.g. 0.5 to 18.5% by volume, preferably 1 to 10% by volume, e.g. 2 to 7% by volume, 3 to 6% by volume, 3 to 7% by volume, 4 to 5% by volume, 4 to 6% by volume, 5 to 8% by volume, 5 to 10% by volume, 6 to 7% by volume or 6 to 8% by volume, more preferably 5 to 8% by volume or 5 to 10% by volume, e.g. 6 to 7% by volume or 6 to 8% by volume. In the present invention, the effect of improving depressive symptoms tends to be enhanced as the hydrogen gas concentration increases within the range not exceeding the explosion limit.

Since hydrogen is a combustible and explosive gas, it is preferable that hydrogen be administered to a human while being contained in the composition of the present invention under safe conditions for humans in the improvement of depressive symptoms.

When the gas other than hydrogen gas is air, the concentration of the air is in the range of, for example, 81.5 to 99.5% by volume.

When the gas other than hydrogen gas is a gas containing oxygen gas, the concentration of the oxygen gas is in the range of, for example, 21 to 99.5% by volume.

The composition may contain nitrogen gas as another main gas. The composition may contain a gas present in the air, such as carbon dioxide, in an amount equivalent to the abundance in the air.

The composition of the present invention may be administered to a human in combination with a therapeutic drug for depressive symptoms, e.g. an antidepressant drug or a mood-stabilizing drug. As for the time (timing) of the combined administration, they may be administered simultaneously or in series.

Examples of the antidepressant drug include, but are not limited to, vortioxetine, escitalopram (Lexapro™), bupropion, mirtazapine (Reflex™, Remeron™), amitriptyline (Tryptanol™), agomelatine, paroxetine (Paxil™), venlafaxine (Effexor™), duloxetine (Cymbalta™), milnacipran (Toledomin™), aripiprazole (Abilify™) and bromazepam (Lexotan™).

The mood-stabilizing drug is a drug serving as a base of drug therapy for disorders accompanying change in mood (dysthymia), for example, bipolar disorder, and examples thereof include, but are not limited to, lithium carbonate (Limas™), carbamazepine (CBZ), sodium valproate (Depakene™, Selenica™, Valerin™), lamotrigine (Lamictal™) and aripiprazole (Abilify™).

The hydrogen gas-containing gas is blended with air or oxygen in such a manner as to obtain a predetermined hydrogen gas concentration, and then filled into, for example, a vessel with pressure resistance (e.g. a cylinder made of metal such as stainless steel). Alternatively, the hydrogen gas-containing gas may be prepared in situ using a hydrogen gas generating apparatus, e.g. a known or commercially available hydrogen gas supplying apparatus (apparatus for generating a hydrogen gas-containing gas), at the time of administration.

By the hydrogen gas generating apparatus, hydrogen gas generated by reaction of a hydrogen generator (e.g. metallic aluminum, magnesium hydride, etc.) with water can be mixed with a diluting gas (e.g. air, oxygen, etc.) at a predetermined ratio (Japanese Patent No. 5228142, etc.). Alternatively, hydrogen gas generated by electrolysis of water is mixed with a diluting gas such as oxygen or air (Japanese Patent No. 5502973, Japanese Patent No. 5900688, etc.). By such an apparatus, a hydrogen gas-containing gas having a hydrogen concentration within the range of 0.5 to 18.5% by volume can be prepared. Examples of the hydrogen gas supplying apparatus include MHG-2000™ and MHG-2000α™ (manufactured by MiZ Company Limited), and by these apparatuses, hydrogen gas-containing air having a hydrogen gas concentration of 3.0 to 10.0% by volume can be prepared on site.

A hydrogen gas-containing gas prepared using an apparatus as described above is, for example, parenterally administered to a human having depressive symptoms.

Other forms of the composition of the present invention include dosage forms (e.g. tablets, capsules, etc.) which are prepared for oral administration to (or ingestion by) a human and which contain a hydrogen generator enabling generation of hydrogen in the gastrointestinal tract. It is preferable that the hydrogen generator be composed of ingredients approved as, for example, food or food additives.

### 3. Improvement of Depressive Symptoms

A second aspect of the present invention provides a method for improving depressive symptoms in a human having depressive symptoms, the method comprising administering the composition of the present invention to the human.

The method for administering the composition of the present invention to a human is preferably pulmonary administration through, for example, inhalation or aspiration. In inhalation of gas, the gas can be inhaled from the mouth or the nose through a nasal cannula or a mask-shaped device covering the mouth and the nose, sent to the lung, and delivered to every part of the body through blood.

The hydrogen gas-containing gas having the above-described hydrogen concentration can be administered to a human one or more times (e.g. 2 or 3 times) a day over a period of 1 week to 3 months or more, e.g. 1 week to 6 months or more. The hydrogen gas-containing gas can be administered over, for example, 10 minutes to 2 hours or more, 30 minutes to 60 minutes or more, 30 minutes to 2 hours or more, 3 hours or more, 4 hours or more or 6 hours or more per dose or per day. In pulmonary administration of the hydrogen gas-containing gas through inhalation or aspiration, the gas can be administered to a human in an atmospheric-pressure environment, or in a high-pressure environment at a high pressure within the range exceeding standard atmospheric pressure (about 1.013 atm) and not exceeding 7.0 atm, e.g. 1.02 to 7.0 atm, preferably 1.02 to 5.0 atm, more preferably 1.02 to 4.0 atm, still more preferably 1.02 to 1.35 atm. Systemic absorption of hydrogen in a human can be promoted by administration in a high-pressure environment.

The high-pressure environment can be created by using a high-pressure housing (e.g. capsule-like housing) designed to have sufficient strength so that a high pressure exceeding standard atmosphere and not exceeding 7.0 atm can be produced in the housing by, for example, injecting the hydrogen gas-containing gas (e.g. hydrogen gas-containing oxygen or air). The shape of the high-pressure housing is preferably nonangular and rounded in whole for attaining pressure resistance. The material of the high-pressure housing is preferably light in weight and high in strength, and examples thereof include reinforced plastics, carbon fiber composite materials, titanium alloys and aluminum alloys. A human can be given a composition containing hydrogen gas together with oxygen gas or air in the high-pressure housing.

It is desirable that a hydrogen gas generating apparatus (e.g. hydrogen gas supplying apparatus (or gaseous hydrogen inhaling apparatus) confirmed to have sufficient therapeutic effect and safety be used in the treatment with the composition of the present invention.

### EXAMPLE

The present invention will be described in further detail by way of Example below, which should not be construed as limiting the technical scope of the present invention.

### [Example 1] Case of Improvement of Depressive Symptoms by Aspiration (or Inhalation) of Hydrogen

A patient (female in her thirties) diagnosed as having depression, who had suffered from anthropophobia and insomnia along with depressive symptoms, received intramuscular injections of Abilify™ (Otsuka Pharmaceutical Co., Ltd. (Japan); with indication of depressive disorder) prescribed by a doctor at the department of psychiatry, and took Lexotan™ Tablet 2 (Chugai Pharmaceutical Co., Ltd. (Japan); with an indications of anxiety and tension associated with depressive disorder) and Brotizolam Tablet (Choseido Pharmaceutical Co., Ltd.; with indication of insomnia); however, the symptoms were not improved. Thus, hydrogen gas (70 to 140 ml/min) was aspirated for 1 hour once or twice a day using a hydrogen aspirator (MHG-2000α™ (MiZ Company Limited)) over about half a year while the medication was continued. As a result, the number of days on which it was possible to naturally fall asleep increased (2 or 3 days a week), and the liveliness of voice was enhanced. About half a year after the start of aspiration (or inhalation) of hydrogen gas, administration of the Brotizolam Tablet was discontinued, and the amount of hydrogen gas aspirated was increased (280 ml/min for 6 hours per day). The aspiration of hydrogen gas was continued over about 3 months. As a result, the depressive symptoms were improved, and therefore the use of Abilify and Lexotan Tablet 2 was discontinued on the basis of the doctor's judgement. The improving effect on depressive symptoms is enhanced as the amount of hydrogen gas aspirated by the patient per day increases. After the use of Abilify and Lexotan Tablet 2, aspiration of hydrogen gas (280 ml/min for 6 hours per day) was continued. As a result, the care personnel evaluated the patient as “being much more cheerful in disposition than before”. The depressive symptoms were improved to the extent that the patient went outside more frequently than before, and enjoyed a life similar to a healthy person.

### INDUSTRIAL APPLICABILITY

According to the present invention, depressive symptoms can be improved by administering hydrogen to a human having depressive symptoms. Since hydrogen itself is not known to have adverse reactions, the QOL of a patient can be improved in treatment of a disease associated with depressive symptoms, such as depression.

1. A composition for improving depressive symptoms in a human, comprising hydrogen as an active ingredient.

2. The composition according to claim 1, wherein the depressive symptoms are caused by a disease selected from the group consisting of depression, bipolar disorder, dysthymic disorder, anxiety disorder, social anxiety disorder, panic disorder, obsessive-compulsive disorder, autism spectrum disorder, ADHD, sleep disorder, cerebral infarction, dementia, alcohol dependence, and an adverse reaction to a therapeutic drug.

3. The composition according to claim 1, wherein the composition is in the form of a hydrogen gas-containing gas.

4. The composition according to claim 3, wherein the hydrogen gas-containing gas has a hydrogen concentration of 5 to 10% by volume.

5. The composition according to claim 1, wherein the composition is administered to a human by pulmonary administration.

6. The composition according to claim 5, wherein the pulmonary administration is performed in an atmospheric-pressure environment or in a high-pressure environment at 1.02 to 7.0 atm.

7. The composition according to claim 1, wherein the composition is prepared in situ using a hydrogen gas generating apparatus at the time of administering the composition to the human.

8. The composition according to claim 1, wherein the composition is administered in combination with a therapeutic agent(s) for the depressive symptoms.

9. The composition according to claim 8, wherein the therapeutic drug(s) is an antidepressant drug or a mood-stabilizing drug.

10. A method for improving depressive symptoms in a human having depressive symptoms, comprising administering to the human the composition according to any one of claims 1 to 9.
